(19) United States
(12) Patent Application Publication
(10) Pub. No.: US 2011/0046460 A1
(43) Pub. Date: Feb. 24, 2011

(54) METHOD AND MEASUREMENT DEVICE FOR RECORDING MEASUREMENT SIGNALS FROM VITAL TISSUE

(76) Inventors: Holger Jungmann, Gelsenkirchen (DE); Michael Schietzel, Herdecke (DE)

Correspondence Address: KF ROSS PC, 5683 RIVERDALE AVENUE, SUITE 203 BOX 900, BRONX, NY, 10471-0900, US

(21) Appl. No.: 12/866,042

(22) PCT Filed: Feb. 25, 2009
(86) PCT No.: PCT/EP09/01340

§ 371 Date: Nov. 15, 2010

(30) Foreign Application Priority Data
Feb. 25, 2008 (DE) 10 2008 011 245.3

(51) Int. Cl. A61B 5/1455

(52) U.S. Cl. 600/322

(57) ABSTRACT

The invention is directed to a measuring method and apparatus for taking spectrometric readings from vital tissue. The object of the invention is to provide solutions by which, in the course of a spectrometric measurement, it is possible to generate measurement values that, compared to above-described previous recording methods, provide more comprehensive information. According to the invention, this object is attained by a method that generates spectrometric signals and in which light is directed into a sample of vital tissue to be examined, remission light exiting the tissue sample to be examined is fed to a spectrometer, and the spectrometer generates output signals that represent the intensity of the remission light with respect to the wavelength, the measurement being performed in such a manner that it extends over a period of time during which the presence of hemoglobin in the examined tissue sample is actively changed by changing pressure on the tissue, wherein the spectra determined in succession for different tissue pressures are used in order to determine changes in the spectra induced by the hemoglobin concentration, and, from these different spectra, the concentrations of selected substances in the tissue sample are calculated. In this manner, it is advantageously possible to generate, in relatively quick succession, a plurality of spectra relating to the tissue sample under examination, wherein these spectra have differences (“distortions”) caused by actively induced changes of pressure on the tissue, and the differences are sufficient for determining the concentration of substances to be detected in the vascular parts of the tissue system.

The invention relates to a method and apparatus for taking readings from vital tissue, in particular for determining the composition of tissue that conveys intravascular hemoglobin.

Measurement methods are known where an analysis of vital tissue is carried out by placing a mobile spectrometer on a tissue sample, and the spectrum of remission light exiting the tissue is recorded by the mobile spectrometer. Based on the spectrum recorded in such a manner, the different substances present in the tissue sample being examined can be identified.

The object of the invention is to provide a solution by which in the course of a spectrometric measurement, measuring values can be generated that provide more accurate information, in particular with respect to the presence or concentration of substances in a vascular tissue sample.

This object is attained according to the invention by a method of taking spectrometric readings in which:
light is directed into a sample of vital tissue to be examined,
remission light exiting from the tissue sample being examined is fed to a spectrometer, and
the spectrometer generates output signals that represent the intensity of the remission light with respect to the wavelength,
the measurement is performed in such a manner that it extends over a period of time (T) during which the presence of hemoglobin in the examined tissue sample is actively changed by changing pressure on the tissue,
the succession of spectra determined for different tissue pressures is used to determine changes in the spectra induced by the hemoglobin concentration, and
from these different spectra, the concentrations of selected substances in the tissue sample are calculated.

This way, it is possible in an advantageous manner to generate a relatively quick succession of spectra related to the tissue sample being examined, these spectra having differences (“distortions”) caused by actively induced changes of pressure on the tissue, which differences make it possible to determine the concentration of quasi-stationary substances to be detected in the vascular parts of the tissue sample being examined.

According to a particularly preferred embodiment of the invention, the active change of the pressure applied to the tissue sample being examined is done by applying pressure to the tissue sample with a gaseous pressure medium.

The pressure medium, in particular ambient air, is preferably applied by an elastomeric cup connected to an optical measuring head. The superatmospheric pressure can be generated here by elastic deformation and compression of the enclosed air. The pressures created in this manner can be detected by a pressure sensor integrated in the measuring head.

It is possible to design the elastomeric cup in such a manner that when applying it and with stepwise increasing pressure levels at each of which spectrometric signals are generated. When applying the elastomeric cup and compressing it by approximately 30 mm, a pressure range of 1000 to approximately 1700 bar can be traversed. For this pressure range, a respective spectrum is preferably recorded at pressure intervals of 50 bar,

The pressure change can also be generated in a different manner, for example purely mechanically by pressing on a window element, preferably at the same time as a pressure measurement, or by supplying pressurized gas, or also by suction, in particular by a heating light source.

The light source is preferably integrated into the measuring head. The spectrum of the light generated by the light source is preferably adjusted in such a manner that the substances to be detected are made to fluoresce. For the detection of blood components, light in the near infrared range is particularly suitable.

Further details and features of the invention are seen in the following description with reference to the drawing. Therein:

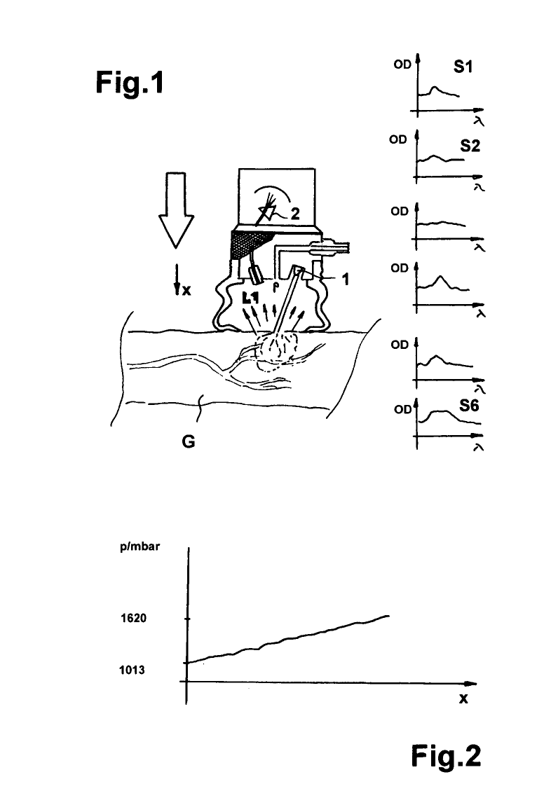

FIG. 1 is a diagram illustrating a measuring system according to the invention for successively generating substance spectra while gradually increasing the pressure on the tissue sample to be examined,

FIG. 2 is a diagram illustrating pressure on the tissue under increasing compression of an elastomeric cup.

FIG. 1 shows greatly simplified a measuring system for generating spectrometric readings. The measurement system comprises a light source 1, preferably an LED, and a receiver 2 that is shown only for illustration purposes as a prism and that can capture light L1 exiting from a sample G of vital tissue.

The receiver 2 comprises a spectrometer by means of which output signals are generated that represent the intensity of the remission light L1 at each of a plurality of different wavelengths. The measuring system according to the invention is operated in such a manner that over a relative short period of time, the pressure p on the tissue sample to be examined is increased, so that as the pressure p in the tissue increases, a plurality of spectra S1, S2 . . . , S6 are recorded.

From the spectra successively recorded for different tissue pressures and by using a correlation approach, the concentration of certain substances in the vascular parts of the tissue that are less pressure sensitive with respect to their composition can be calculated.

The calculation of the concentration of the substances is carried out by using a phenomenon that is characterized in that by changing pressure on the tissue, the concentration of hemoglobin in this tissue is changed. As a result of the change of the concentration of a substance whose spectral properties are known, it is possible to determine the (largely unchanged) concentrations of the other substances contained in a quasi-stationary manner in the tissue sample. If the concentration of hemoglobin included in the sample changes, the absorption contribution of the substance changes depending on the concentration, in particular in the near and mid infrared range. This effect is used according to the invention to quantify the substances contained in a quasi-stationary manner in the tissue being examined. According to the invention, the pressure applied to the tissue being examined is changed to change the concentration of substances with known spectral properties and, at the same time, the absorption spectra for a plurality of pressure levels that are relatively closely spaced relative to one another are determined.

Let μa=Σciεi be the absorption coefficient of the hemoglobin. The reduced scattering coefficient of the tissue in which the hemoglobin is embedded is μs. If A=log(R0/Rm) is the measured skin spectrum, then: μa and μs proportional to A and A=f*μa+μs. If μa is changed, for example by pressure, μs remains constant. From Ai=f*μa+μs, μs can be extrapolated. With a known μs, the concentration of hemoglobin can be determined iteratively by the diffusion theory.

1. A method of generating spectrometric readings, the method comprising the steps of:
a) directing light into a sample of vital tissue to be examined,
b) feeding remission light exiting the tissue sample to be examined to a spectrometer,
c) generating with the spectrometer output spectra that represent the intensity of the remission light with respect to the wavelength,
d) repeating steps a to c over a period of time during which pressure on the tissue is changed to actively alter the presence of hemoglobin in the examined tissue sample, and
e) comparing the spectra determined in succession for different tissue pressures to determine changes in the spectra induced by the hemoglobin concentration, and
f) calculating from these different spectra the concentrations of selected substances in the tissue sample.

2. The method according to claim 1, wherein pressure on the tissue is changed by locally applying pressure with a gaseous medium.

3. The method according to claim 2, wherein the pressure is applied by using an elastic bellows.

4. The method according to claim 3, wherein the bellows is connected to a measuring head that has a light source usable for step a and a light sensor usable for step
b.

5. The method according to claim 1 wherein pressure on the tissue is increased.

6. The method according to claim 1, wherein for each tissue pressure level, a spectrum of the remission light is recorded.

7. The method according to claim 1 wherein for recording the successively recorded spectra, a data field is created for data for each resolved wavelength value and the intensity or optical density and pressure on the tissue.

8. A mobile spectrometer comprising:
a storage device,
an evaluation device,
means for directing light into a sample of vital tissue to be examined,
means for feeding remission light exiting the tissue sample to be examined to a spectrometer,
means for generating output signals that represent the intensity of the remission light with respect to its wavelength,
means for changing the pressure in the tissue sample to be examined, and
means connected to the storage device and to the evaluation device for recording a plurality of spectra detected at different tissue pressures.

9. An apparatus for generating spectrometric readings, the apparatus comprising:
means for directing light into a sample of vital tissue to be examined, whereby remission light is caused to issue from the tissue sample,
a spectrometer;
means for feeding the remission light exiting the tissue sample to the spectrometer,
means for changing pressure on the sample over a predetermined period of time;
means in the spectrometer for generating at different pressure levels within the period of time spectra that represent the intensity of the remission light with respect to the wavelength at the respective pressure level; and
evaluating means for comparing these spectra to each other and determining changes in the spectra and for suppressing the changes in the spectra, whereby only relatively unchanging portions of the spectra relating to permanent substances in the sample remain.
